SEQUENCE LISTING (1 sequence)

SEQ ID NO: 1 - protein, 13 aa, synthetic construct
  source 1..13: note = Lactoferrin polypeptide fragment
KNLLFNDNTECLA

(19) United States
(12) Patent Application Publication
(10) Pub. No.: US 2025/0306014 A1
(43) Pub. Date: Oct. 2, 2025

(54) Lactoferrin Polypeptide Fragment, Preparation Method Thereof, Antibody Prepared Using the Same, and Applications

(71) Applicant: Beijing Biosynthesis Biotechnology Co., Ltd., Beijing (CN)

(72) Inventors: Ju WU, Beijing (CN); Xiaole WU, Beijing (CN); Jianxiang ZHANG, Beijing (CN); Zhijuan JI, Beijing (CN); Fengying ZHANG, Beijing (CN); Junlong ZHANG, Beijing (CN); Haoran WANG, Beijing (CN)

(21) Appl. No.: 18/618,970

(22) Filed: Mar. 27, 2024

(51) Int. Cl. G01N 33/533; C07K 1/04; C07K 1/06; C07K 1/14; C07K 14/79; C07K 16/18

(52) U.S. Cl. CPC G01N 33/533; C07K 1/042; C07K 1/063; C07K 1/145; C07K 14/79; C07K 16/18; C07K 2317/14

(57) ABSTRACT

In the first aspect, the present disclosure provides a Lactoferrin polypeptide fragment, where the Lactoferrin polypeptide fragment has an amino acid sequence shown in SEQ ID NO: 1. In the second aspect, the present disclosure provides a preparation method of the Lactoferrin polypeptide fragment, including: protecting amino acids using a 9-fluorenylmethoxycarbonyl (Fmoc) protecting group, coupling the amino acids with a P-hydroxymethylphenoxymethyl polyethylene resin (HMP resin) in an order of cysteine, alanine, leucine, cysteine, glutamate, threonine, asparagine, aspartate, asparagine, phenylalanine, leucine, leucine, asparagine, and lysine, and separating the HMP resin to obtain a target Lactoferrin polypeptide fragment. In the third aspect, the present disclosure further provides an anti-Lactoferrin antibody prepared using the Lactoferrin polypeptide fragment, and use of the Lactoferrin polypeptide fragment in preparation of an anti-Lactoferrin flow cytometric antibody and in preparation of a Lactoferrin detection product.

### TECHNICAL FIELD

The present disclosure relates to the technical field of biomedicine, and in particular to a Lactoferrin polypeptide fragment, a preparation method thereof, an antibody prepared using the same, and applications.

### REFERENCE TO SEQUENCE LISTING

A computer readable XML file entitled “GWP20240100834_seqlist”, that was created on Mar. 22, 2024, with a file size of about 1,775 bytes, contains the sequence listing for this application, has been filed with this application, and is hereby incorporated by reference in its entirety.

### BACKGROUND

Lactoferrin, widely distributed in human and mammalian milk and various other tissues and tissue fluids, is an iron-binding glycoprotein with a molecular weight of approximately 80 kDa as well as a member of the transferrin family. Lactoferrin is highly expressed in neutrophils and has a wide range of biological activities, including broad-spectrum antibacterial effects, anti-inflammation, inhibition of tumor cell growth, and regulation of the body's immune response. Lactoferrin is considered a new type of antibacterial and anticancer drug and a food and cosmetic additive with great development potential. For example, the Food and Drug Administration (FDA) in USA has already allowed the Lactoferrin to be used as a food additive for sports and functional foods. Lactoferrin acts as a component of the innate immune system. In addition to its primary function of being able to bind and transport iron ions, the Lactoferrin also shows antiviral, antiparasitic, catalytic, antiallergic, and radioprotective functions and properties. As the application of Lactoferrin becomes increasingly widespread, a demand is also growing for qualitative and quantitative detection of the Lactoferrin.

At present, immunological detection is one of the most commonly used methods for qualitative and quantitative detection of Lactoferrin in samples, and has been applied to a certain extent in the fields of scientific research and food processing. However, as a core component of immunological detection, anti-Lactoferrin antibodies generally have problems such as low titer and poor sensitivity. In addition, most of the antibodies commonly used in flow cytometry are obtained by immunizing animals with cells or cell extracts. Although the cells can be identified, their antigenic epitope is unclear and the theoretical basis of the experimental data is inaccurate. As a result, a preparation method of these antibodies has a complicated process with poor repeatability, and is difficult to control manually. Therefore, the antibodies produced have variations, and non-specific reactions of such antibodies may cause adverse effects on later experiments.

### SUMMARY

In order to solve at least one of the above technical problems, develop an immunogen, and then provide a basis for preparing Lactoferrin-specific antibodies, the present disclosure provides a Lactoferrin polypeptide fragment, a preparation method, an antibody prepared using the same, and applications.

In the first aspect, the present disclosure provides a Lactoferrin polypeptide fragment, where the Lactoferrin polypeptide fragment has an amino acid sequence shown in SEQ ID NO: 1.

The Lactoferrin polypeptide fragment designed by adopting the above technical solutions is highly hydrophilic, easy to synthesize and purify, and is suitable for mass production. Moreover, the Lactoferrin polypeptide fragment has strong immunogenicity, such that an antibody prepared using the Lactoferrin polypeptide fragment has desirable specificity, high sensitivity, and clear and sharp WB bands, and is extremely suitable for use in flow cytometry (FC) detection of human tissue lymphoma cells (U-937).

In the second aspect, the present disclosure provides a preparation method of the Lactoferrin polypeptide fragment, including the following steps:
S1, activation: ligating an amino group of an amino acid to a 9-fluorenylmethoxycarbonyl (Fmoc) protecting group through a reaction to obtain an Fmoc-protected amino acid (Fmoc-AA), and activating a carboxyl group of the Fmoc-AA to obtain a carboxyl-activated Fmoc protecting group-containing amino acid (carboxyl-activated Fmoc-AA);
S2, ligation of a resin: subjecting the carboxyl-activated Fmoc-AA obtained in step S1 to a reaction with a P-hydroxymethylphenoxymethyl polyethylene resin (HMP resin) to obtain a carboxyl-activated Fmoc-AA-ligated resin;
S3, deprotection: removing an Fmoc protecting group of the carboxyl-activated Fmoc-AA-ligated resin prepared in step S2 through deprotection, such that an amino group of a remaining amino acid is activated to obtain an amino-activated resin;
S4, conducting coupling: repeating step S1 to prepare a new carboxyl-activated Fmoc-AA, and coupling the amino-activated resin prepared in step S3 with the new carboxyl-activated Fmoc-AA to obtain a new amino acid-coupled and Fmoc protecting group-protected amino acid-containing resin;
S5, preparation of a polypeptide resin: conducting a process of repeating step S3 to remove the Fmoc protecting group from the new amino acid-coupled and Fmoc protecting group-protected amino acid-containing resin prepared in step S4 and then repeating step S4 to couple a new amino acid to a resulting product, repeating S1 to S4, and repeating step S3 to remove the Fmoc protecting group until a target Lactoferrin polypeptide fragment-ligated resin is obtained; and
S6, conducting separation and purification: separating a resin from the target Lactoferrin polypeptide fragment-ligated resin prepared in step S5 to obtain a crude Lactoferrin polypeptide fragment, and purifying the crude Lactoferrin polypeptide fragment to obtain the Lactoferrin polypeptide fragment; where
the amino acids in steps S1 to S5 are added in an order of alanine, leucine, cysteine, glutamic acid, threonine, asparagine, aspartate, asparagine, phenylalanine, leucine, leucine, asparagine, and lysine.

By adopting the above technical solutions, the Lactoferrin polypeptide fragment of the present disclosure can be easily produced, and the prepared polypeptide fragment has an accurate structure, a short preparation cycle, and a low cost.

Optionally, synthesis in steps S1 to S5 is completed using an automated polypeptide synthesizer.

By adopting the above technical solutions, the preparation process can be made simpler, the polypeptide fragment has a more accurate structure, and the preparation method has the advantages of high coupling rate, short cycle, and extremely high degree of automation, and the preparation cost can be effectively reduced.

Optionally, a process of activating the carboxyl group in step S1 includes subjecting the Fmoc-AA to a reaction with N,N-dicyclohexylcarbodiimide (DCC) and 1-hydroxybenzotriazole (HOBT), and has a reaction formula as follows:

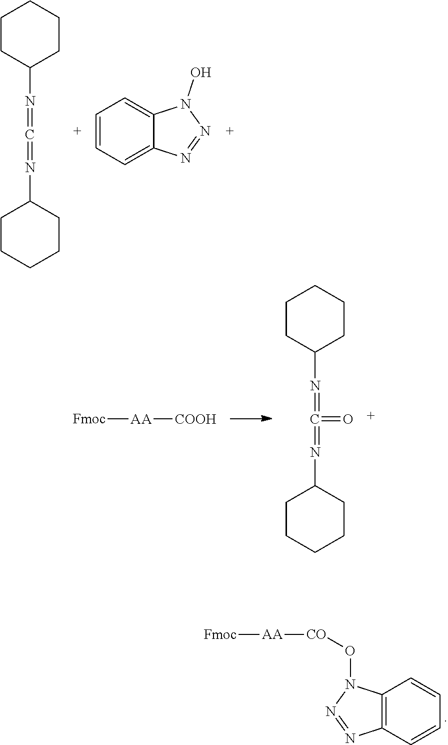

By adopting the above technical solutions, the activation has mild conditions and is easy to activate the carboxyl group, and the combination of DCC and HOBT can also effectively inhibit racemization in peptide synthesis.

Optionally, a process of subjecting the carboxyl-activated Fmoc-AA to the reaction with the HMP resin in step S2 is conducted in the presence of dimethylaminopyridine (DMAP), and has a reaction formula as follows:

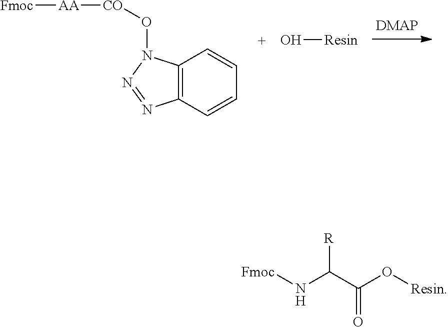

By adopting the above technical solutions, the reaction conditions are relatively mild, the reaction yield is high, the resin connection site is accurate, and there are few side reactions, thereby effectively improving the purity of the finally prepared polypeptide fragment.

Optionally, a process of removing the Fmoc protecting group in step S3 is conducted under the action of Piperidine, and has a reaction formula as follows:

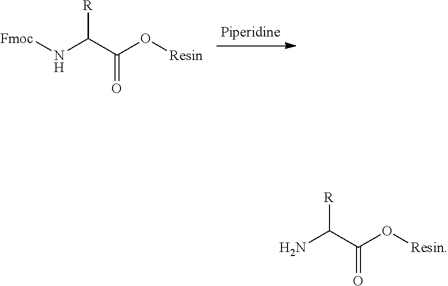

By adopting the above technical solutions, using Piperidine to cut the Fmoc protecting group has the best cutting effect; and after the reaction, draining the resin and washing the resin can remove excess Piperidine and reaction by-products, making it easy to operate.

Optionally, the coupling in step S4 refers to a coupling reaction with a reaction formula as follows:

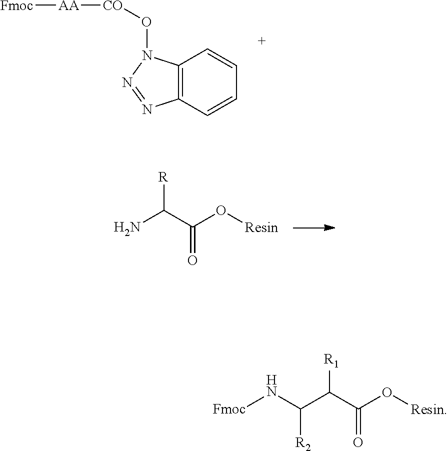

By adopting the above technical solutions, the coupling effect is better, the coupling rate is extremely high, there are very few side reactions, and the reaction rate is also high, thereby effectively shortening a synthesis cycle and improving a purity of the polypeptide fragment.

Optionally, a process of separating the resin from the target Lactoferrin polypeptide fragment-ligated resin in step S6 includes adding trifluoroacetic acid (TFA) combined with a scavenger mixed with 1,2-ethanedithiol (EDT), thioanisole, and water to allow a reaction with the target Lactoferrin polypeptide fragment-ligated resin to separate the target Lactoferrin polypeptide fragment from the resin.

By adopting the above technical solutions, the reaction yield is higher, the separation effect is better, the reaction conditions are mild, and the scavenger used is easier to remove.

Optionally, after the target Lactoferrin polypeptide fragment and the resin are separated, the resin is removed by filtration while the scavenger is removed by vacuum distillation; and a resulting residue is subjected to water dissolution and extraction in sequence to obtain the crude Lactoferrin polypeptide fragment.

By adopting the above technical solutions, the separated resin and other reagents can be effectively removed to obtain a crude Lactoferrin polypeptide fragment with a lower impurity content, thereby effectively reducing a difficulty of purification and effectively improving a purity of the purified Lactoferrin polypeptide fragment.

Optionally, an extractant for the extraction is diethyl ether.

By adopting the above technical solutions, choosing diethyl ether as the extractant can effectively reduce costs.

Optionally, the crude Lactoferrin polypeptide fragment in step S6 is purified by high-performance liquid chromatography (HPLC).

By adopting the above technical solutions, HPLC has the best purification effect and the produced Lactoferrin polypeptide fragment has higher purity.

Optionally, the HPLC includes:
chromatographic column: C₁₈ 25 mm×250 mm;
mobile phase A: 0.1% TFA aqueous solution; mobile phase B: 0.1% TFA dissolved in 60% acetonitrile;
detection wavelength: 214 nm;
flow rate: 10 mL/min; and
elution gradient: 20% to 60% of the mobile phase B for a total of 30 min.

By adopting the above technical solutions, excellent purification effects can be obtained, and the purity of the Lactoferrin polypeptide fragment obtained after purification can reach more than 98%.

In the third aspect, the present disclosure further provides an anti-Lactoferrin antibody prepared using the Lactoferrin polypeptide fragment, where the anti-Lactoferrin antibody is prepared by: coupling the Lactoferrin polypeptide fragment to a carrier protein, conducting rabbit immunization, collecting rabbit blood when a specific immunoglobulin (IgG) concentration in a rabbit serum reaches a peak, and then separating and purifying the rabbit serum for the anti-Lactoferrin antibody.

By adopting the above technical solutions, the rabbit antibody prepared has strong specificity, high sensitivity, and desirable stability; the rabbit antibody can also efficiently bind to Lactoferrin-positive cells and can be used to sort cells with differential Lactoferrin expression.

Optionally, the carrier protein is selected from the group consisting of hemocyanin and keyhole limpet hemocyanin.

By adopting the above technical solutions, a complete immunogen can be produced after connecting the carrier protein, and has a better immune effect.

Optionally, the rabbit immunization is conducted by basal immunization and/or multiple booster immunizations.

By adopting the above technical solutions, a better immune effect and a high-titer antibody can be obtained.

In the fourth aspect, the present disclosure further provides use of the Lactoferrin polypeptide fragment in preparation of an anti-Lactoferrin flow cytometric antibody and in preparation of a Lactoferrin detection product.

By adopting the above technical solutions, the excellent characteristics of the Lactoferrin polypeptide fragment of the present disclosure can be acquired to prepare anti-Lactoferrin antibody which is qualified for flow cytometry application. The prepared antibodies have strong specificity, high sensitivity and desirable stability; when being used to prepare Lactoferrin immunobloting products, the products show clear and sharp WB bands, and high sensitivity and accuracy.

To sum up, the present disclosure includes at least one of the following beneficial technical effects:

1. The present disclosure designs a highly ideal Lactoferrin immunogenic polypeptide fragment, which provides a linear epitope of Lactoferrin protein. Therefore, the antibody prepared from the Lactoferrin polypeptide fragment of the present disclosure can recognize the surface protein of living cells, and can be used for flow cytometry detection and WB detection of Lactoferrin protein, with a wide range of applications.

2. The present disclosure designs multiple antigenic epitopes based on the structure and function of Lactoferrin protein. A solid-phase peptide synthesis method is adopted to synthesize peptides separately and couple the same to carrier proteins to complete the preparation of immugens, respectively. Animals are immunized and the best epitopes are selected according to the experimental results. The anti-Lactoferrin antibody prepared using the Lactoferrin immunogen polypeptide fragment has strong specificity, high sensitivity, and desirable stability.

3. In the present disclosure, the preparation method has a simple process that can be conducted using an automated polypeptide synthesizer. The prepared polypeptide fragment has an accurate structure, high purity, short preparation cycle, and low preparation cost.

4. In the present disclosure, a Lactoferrin rabbit antibody prepared using the Lactoferrin immunogenic polypeptide fragment can efficiently bind to Lactoferrin-positive cells and can be used to sort cells with differential Lactoferrin expression.

### BRIEF DESCRIPTION OF THE DRAWINGS

FIG. 1 shows a schematic diagram of HPLC analysis of the Lactoferrin-KA13 polypeptide in an example of the present disclosure;

FIG. 2 shows a WB schematic diagram of the anti-Lactoferrin antibody in detecting breast milk in an example of the present disclosure;

FIG. 3 shows a WB schematic diagram of the anti-Lactoferrin antibody in detecting recombinant proteins in an example of the present disclosure;

FIGS. 4A-4B show schematic diagram of the cell selection range for FC detection of U-937 using the anti-Lactoferrin antibody and a control antibody in an example of the present disclosure;

FIGS. 5A-5B show scatter plots of cell fluorescence distribution of the anti-Lactoferrin antibody and the control antibody for FC detection of U-937 in an example of the present disclosure; and

FIG. 6 shows a fluorescence intensity distribution curve of each group of cells using the anti-Lactoferrin antibody and the control antibody for FC detection of U-937 in an example of the present disclosure; where

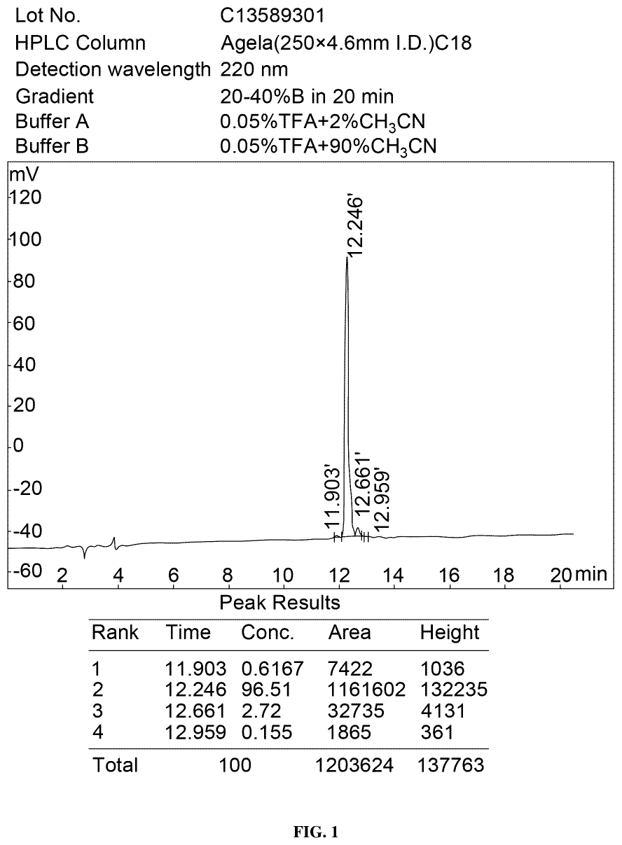

in FIG. 1, HPLC Column: high-performance liquid chromatography column; Detection wavelength: detection wavelength; Gradient: gradient; Buffer A: buffer A; Buffer B: buffer B;

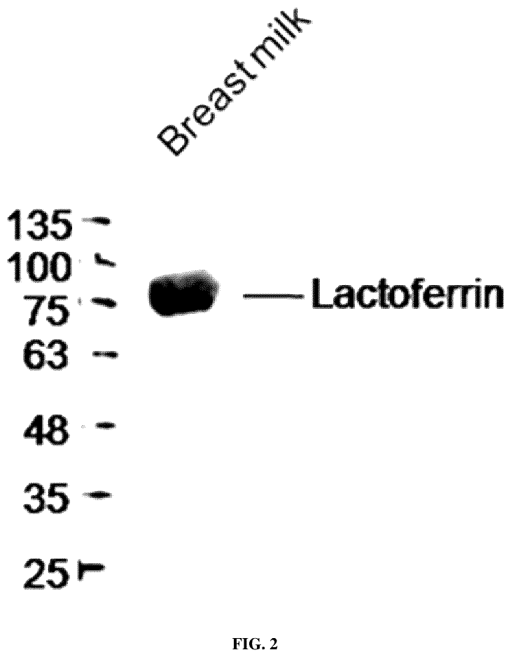

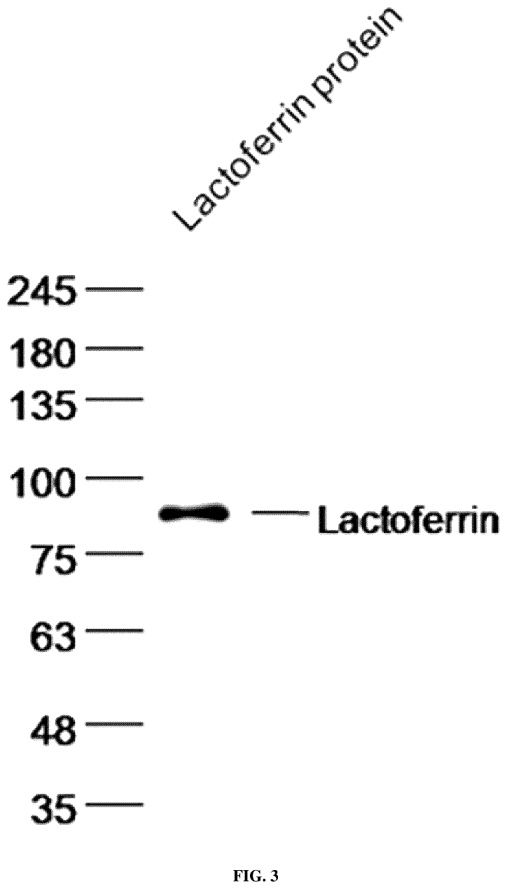

in FIG. 2, Breast milk: breast milk; in FIG. 3, Lactoferrin protein: Lactoferrin recombinant protein; and

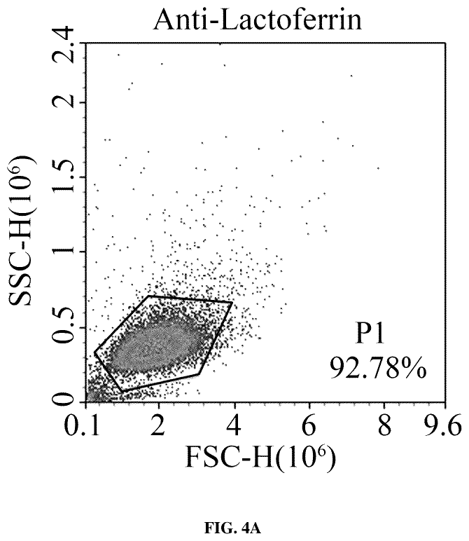

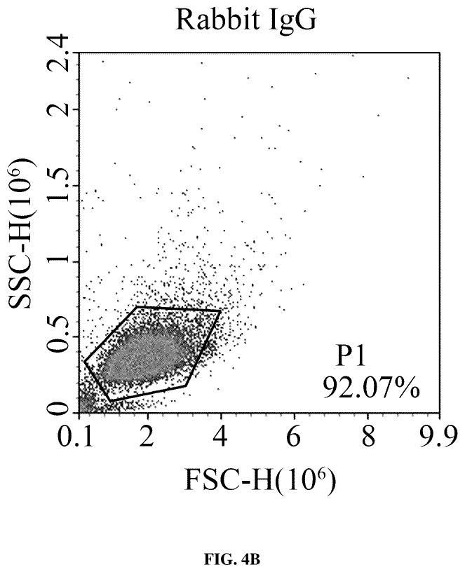

in FIGS. 4A-4B, FIGS. 5A-5B, and FIG. 6, Anti-Lactoferrin: anti-Lactoferrin antibody; Rabbit IgG: rabbit antibody isotype control; Blank: blank control.

### DETAILED DESCRIPTION OF THE EMBODIMENTS

The following further describes the present disclosure with reference to examples and the accompanying drawings.

Explanation of terms: Lactoferrin: lactoferrin protein, Fmoc: 9-fluorenylmethoxycarbonyl, HMP resin: P-hydroxymethylphenoxymethyl polyethylene resin, Resin: resin (specifically the HMP resin); DCC: N,N′-dicyclohexylcarbodiimide, DMAP: 4-dimethylaminopyridine, HOBT: 1-hydroxybenzotriazole, TFA: trifluoroacetic acid, EDT: 1,2-ethanedithiol, Piperidine: piperidine, WB: Western blot, U-937: human tissue lymphoma, FC: flow cytometry.

Experimental materials and reagents required in the examples of the present disclosure:
HMP resin is purchased from CsBio, USA.
Fmoc-protected amino acid (Fmoc-AA) is purchased from Merck, and can also be directly prepared by reacting N-(9-fluorenylmethyloxycarbonyl)oxysuccinimide (Fmoc-OSU) with the desired amino acid.
N-methylpyrrolidone (NMP) is purchased from Merck.
Dichloromethane (DCM) is purchased from Merck.
Methanol (MeOH) is purchased from Merck.
Piperidine is purchased from Merck.
DMAP is purchased from Sigma.
HOBT is purchased from Sigma.
DCC is purchased from Sigma.
TFA is purchased from Sigma.
EDT is purchased from Sigma.
Thioanisole is purchased from Sigma.
Crystallized phenol is purchased from Beijing Chemical Reagent.
Acetonitrile is purchased from Merck.
Experimental instruments required in the examples of the present disclosure:
Automated peptide synthesizer: USA, CsBio 336 multi-channel automated peptide synthesizer;
Rotary evaporator: Japan, YAMATO CE50 type rotary evaporator;
HPLC instrument: Waters 600 type-E;
Freeze dryer: USA, LABCANCO;
Vacuum circulating water pump: Zhengzhou Greatwall Scientific Industrial and Trade Co., Ltd., SH-B type;
Centrifuge: USA, Sigma.

In the present disclosure, a designed Lactoferrin polypeptide fragment has an amino acid sequence shown in SEQ ID NO: 1.

First, the Lactoferrin polypeptide fragment of the present disclosure designs multiple antigenic epitopes based on the structure and function of Lactoferrin protein. The solid-phase peptide synthesis method is adopted to synthesize peptides according to the designed antigenic epitopes, and the peptides are coupled with carrier proteins to complete the preparation of the immunogens. After immunizing animals, the best antigenic epitopes are selected through experiments. Polypeptide fragments are designed based on the selected optimal antigenic epitopes, and the feasibility and difficulty of synthesis of the polypeptide fragments are verified through solid-phase peptide synthesis methods. The Lactoferrin polypeptide fragment of the present disclosure is finally selected.

Compared with the existing technology of directly immunizing animals with Lactoferrin protein antigen to prepare monoclonal or polyclonal antibodies to Lactoferrin, and the use of hybridoma technology to prepare anti-Lactoferrin antibodies, the technical solutions of the present disclosure adopt the best antigen epitope obtained by optimization and screening. The Lactoferrin polypeptide fragment designed based on this antigenic epitope is a more excellent immunogenic polypeptide. The prepared anti-Lactoferrin antibody has obvious advantages over existing antibodies in terms of specificity, sensitivity, and stability.

In the present disclosure, the Lactoferrin polypeptide fragment can be prepared by the following steps:
S1, activation: ligating an amino group of an amino acid to a 9-fluorenylmethoxycarbonyl (Fmoc) protecting group through a reaction to obtain an Fmoc-protected amino acid (Fmoc-AA), and activating a carboxyl group of the Fmoc-AA to obtain a carboxyl-activated Fmoc protecting group-containing amino acid (carboxyl-activated Fmoc-AA);
S2, ligation of a resin: subjecting the carboxyl-activated Fmoc-AA obtained in step S1 to a reaction with a P-hydroxymethylphenoxymethyl polyethylene resin (HMP resin) to obtain a carboxyl-activated Fmoc-AA-ligated resin;
S3, removal of a protecting group: removing an Fmoc protecting group of the carboxyl-activated Fmoc-AA-ligated resin prepared in step S2 through deprotection, such that an amino group of a remaining amino acid is activated to obtain an amino-activated resin;
S4, conducting coupling: repeating step S1 to prepare a new carboxyl-activated Fmoc-AA, and coupling the amino-activated resin prepared in step S3 with the new carboxyl-activated Fmoc-AA to obtain a new amino acid-coupled and Fmoc protecting group-protected amino acid-containing resin;
S5, preparation of a polypeptide resin: conducting a process of repeating step S3 to remove the Fmoc protecting group from the new amino acid-coupled and Fmoc protecting group-protected amino acid-containing resin prepared in step S4 and then repeating step S4 to couple a new amino acid to a resulting product, repeating S1 to S4, and repeating step S3 to remove the Fmoc protecting group until a target Lactoferrin polypeptide fragment-ligated resin is obtained; and
S6, conducting separation and purification: separating a resin from the target Lactoferrin polypeptide fragment-ligated resin prepared in step S5 to obtain a crude Lactoferrin polypeptide fragment, and purifying the crude Lactoferrin polypeptide fragment to obtain the Lactoferrin polypeptide fragment; where
the amino acids in steps S1 to S5 are added in an order of alanine, leucine, cysteine, glutamate, threonine, asparagine, aspartate, asparagine, phenylalanine, leucine, leucine, asparagine, and lysine.

The above preparation method has a simple process that can be conducted using an automated polypeptide synthesizer. The prepared polypeptide fragment has an accurate structure, high purity, short preparation cycle, and low preparation cost.

Examples are shown as follows:

### Example 1 Preparation of a Lactoferrin Polypeptide Fragment

A Lactoferrin polypeptide fragment in this example was prepared using the following steps:

### a) Activation of Fmoc-AA

The Fmoc-AA had a structural formula as follows:

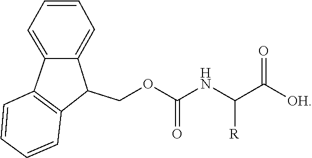

The Fmoc-AA could also be expressed as: Fmoc-AA-COOH.

The Fmoc-AA was activated by reacting with DCC and HOBT to generate carboxyl-activated Fmoc-AA-COOBP, with a reaction formula as follows:

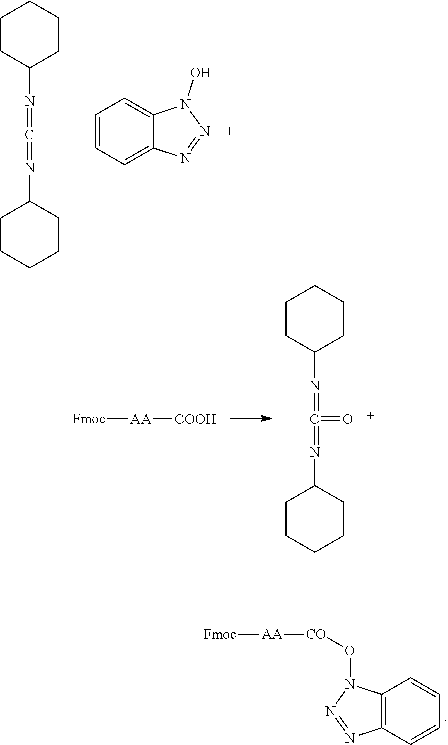

### b) Attaching Activated Amino Acids to the Resin

The activated cysteine and HMP resin were reacted in the presence of DMAP to obtain an amino acid-ligated resin, with a reaction formula as follows:

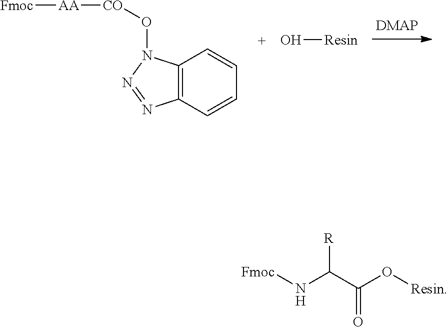

### c) Removing an Fmoc Protecting Group of the Amino Acid-Ligated Resin

The Fmoc protecting group of the amino acid-ligated resin was removed under the action of Piperidine, with a reaction formula as follows:

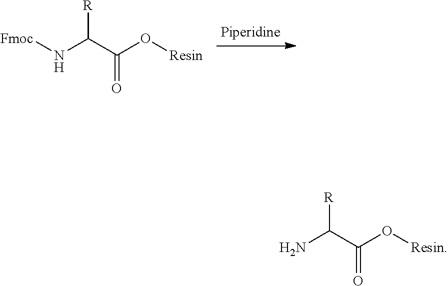

d) Coupling an Fmoc Protecting Group-Removed Resin with Excess Carboxyl-Activated Fmoc-AA

A reaction process of Fmoc-AA carboxyl activation was the same as that in “step a)”. The activated Fmoc-AA was further coupled with the resin obtained in step c), with a reaction formula as follows:

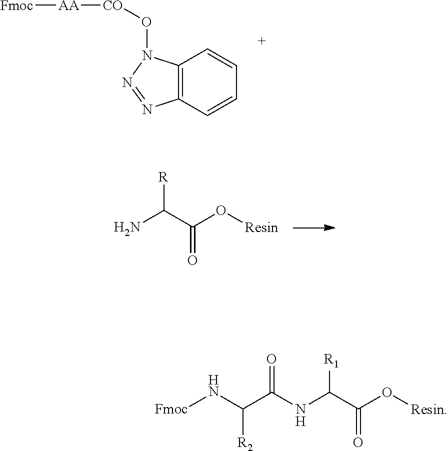

e) Steps c) to step d) were repeated for a total of 13 times to obtain a polypeptide resin, where the order of adding the carboxyl-activated amino acids was: alanine, leucine, cysteine, glutamate, threonine, asparagine, aspartate, asparagine, phenylalanine, leucine, leucine, asparagine, and lysine.

### f) Separation of the Lactoferrin Peptide

TFA combined with a scavenger mixed with EDT, thioanisole, and water were reacted with the obtained polypeptide resin to cleave and separate the Lactoferrin polypeptide fragment from the peptide resin. After the reaction, the resin was removed by filtration and the scavenger was removed by vacuum distillation. The Lactoferrin polypeptide fragment was subjected to water dissolution and diethyl ether extraction to obtain a crude Lactoferrin polypeptide compound.

### g) Separation, Purification, and Analysis of Lactoferrin-KA13 Polypeptide

The crude Lactoferrin polypeptide compound was separated and purified by HPLC.

Specifically:
chromatographic column: C₁₈ 25 mm×250 mm;
mobile phase A: 0.1% TFA aqueous solution; mobile phase B: 0.1% TFA dissolved in 60% acetonitrile;
detection wavelength: 214 nm;
flow rate: 10 mL/min; and
elution gradient: 20% to 60% of the mobile phase B for a total of 30 min.

Thus, a pure Lactoferrin polypeptide fragment was obtained with a purity of over 98%.

The pure Lactoferrin polypeptide fragment obtained after separation and purification was subjected to HPLC analysis to detect a purity. A specific analysis process included:
chromatographic column: C₁₈ 4.6 mm×150 mm;
mobile phase A: 0.1% TFA aqueous solution; mobile phase B: 0.1% TFA dissolved in 60% acetonitrile;
detection wavelength: 214 nm;
flow rate: 1 mL/min; and
elution gradient: 20% to 60% of the mobile phase B for a total of 30 min.

The analysis results were shown in FIG. 1, indicating that the purity of the Lactoferrin polypeptide fragment could reach not less than 98%.

### Example 2 Preparation and Detection of Anti-Lactoferrin Antibody

### I. Materials

The Lactoferrin polypeptide fragment prepared in Example 1;

New Zealand white rabbits for experiment.

### II. Antibody Preparation Steps

Since the Lactoferrin polypeptide fragment prepared in Example 1 did not have complete immunogenicity, there was a demand to be coupled with a carrier protein to prepare a complete immunogen. Therefore, the Lactoferrin polypeptide fragment was first coupled to a protein carrier (hemocyanin or keyhole limpet hemocyanin) to prepare a complete immunogen.

The New Zealand white rabbits for experiment were immunized at multiple points on their backs through basal immunization (complete adjuvant+sufficient emulsification of polypeptide antigen) and multiple booster immunizations (incomplete adjuvant+sufficient emulsification of polypeptide antigen). When a concentration of specific IgG in the serum reached the peak, blood was collected from the rabbit car vein, and the serum was separated to obtain the Lactoferrin rabbit antibody of the present disclosure.

The titer and specificity of the antibody were evaluated using ELISA and WB methods.

After purifying, the IgG was labeled with fluorescein PE, and then the U-937 cells were detected by flow cytometry. Specifically, a U-937 cell suspension was fixated with 50% ethanol, and 2 μL each of PE-labeled anti-Lactoferrin antibody and PE-labeled isotype control IgG were added, while a blank control was added with a same concentration of PE. The flow cytometry detection was conducted after incubation.

### III. Test Results

Detection of antibody sensitivity by ELISA method: the test results showed that the anti-Lactoferrin antibody had a titer of better than 1:32000, indicating that the antibody had high sensitivity.

The specificity of the antibody was detected by WB method, and the results were shown in FIG. 2 and FIG. 3.

As shown in FIG. 2, the Lactoferrin protein had a molecular weight of approximately 80 kDa. As shown in FIG. 3, when the anti-Lactoferrin antibody prepared by the present disclosure was used for WB detection of recombinant protein or breast milk samples, the bands were all located at 75 kDa to 100 kDa, which was consistent with the theoretical molecular weight and had no non-specific bands, proving that the antibody showed an excellent specificity.

The ability of the anti-Lactoferrin antibody to label cells was tested by FC. The results were shown in FIGS. 4A-4B to FIG. 6.

FIGS. 4A-4B showed a cell selection range of the experimental group and the isotype control group.

X-coordinate axis: FSC-H was forward angle detection, to select cells based on their size.

Y coordinate axis: SSC-H was lateral angle detection, to select cells based on intracellular particles.

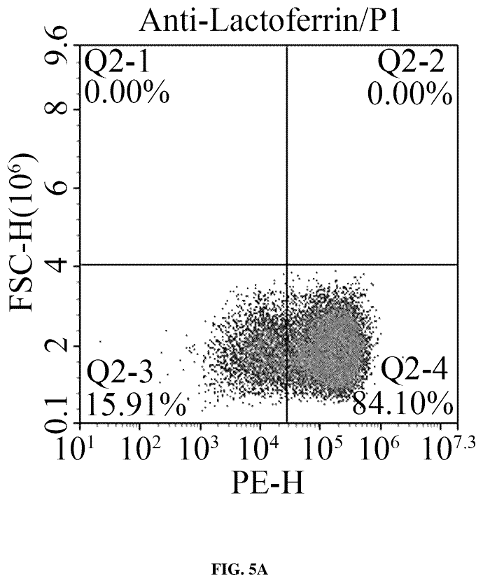

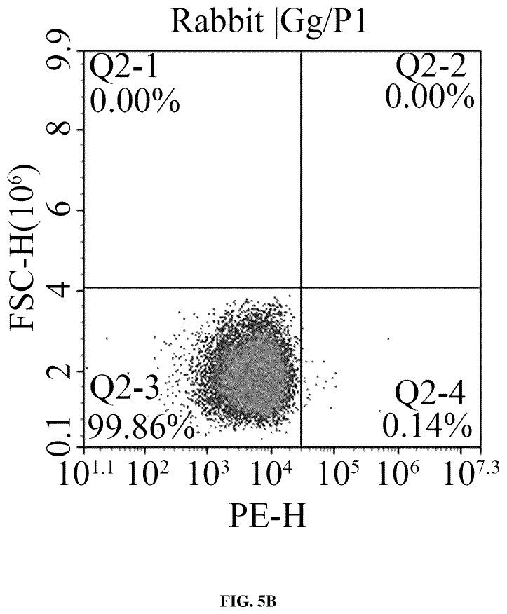

FIGS. 5A-5B showed the scatter plots of cell fluorescence distribution in the experimental group and the isotype control group.

X-coordinate axis: fluorescence intensity of PE.

Y coordinate axis: count of cells under specific fluorescence intensity.

The results showed that PE-labeled anti-Lactoferrin antibody could label 84.1% of cells as positive, and the positive rate of isotype control under the same conditions was about 0.1%. This indicated that the anti-Lactoferrin antibody could be used for FC detection, and the antigen-antibody binding was stable and the labeling effect was excellent.

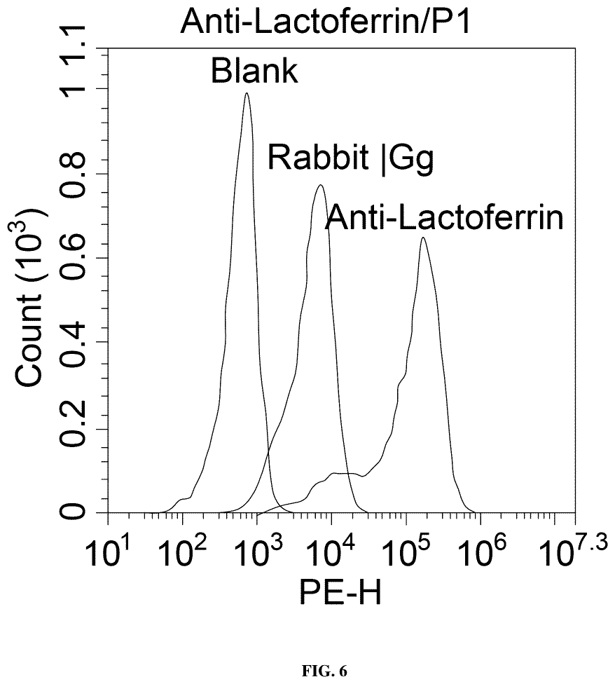

FIG. 6 showed the curve of fluorescence intensity distribution of cells in each group, and the group represented by each curve was shown in the legend.

X-coordinate axis: fluorescence intensity of PE.

Y-coordinate axis: count of cells within a specific fluorescence intensity.

The results showed that the addition of PE-labeled anti-Lactoferrin antibody caused the cell number peak to appear at a position with higher fluorescence intensity. Compared with the isotype control, the peak value of the curve of the experimental group shifted to the right by about 2 orders of magnitude, indicating that the anti-Lactoferrin antibody could efficiently bind to Lactoferrin-positive cells and could be used to sort cells with differential Lactoferrin expression.

According to the above examples of the present disclosure, it can be seen that the present disclosure has established a highly ideal immunogenic polypeptide and successfully prepared an antibody that can be used for Lactoferrin detection. That is: based on the structure and function of Lactoferrin protein, multiple antigenic epitopes are analyzed and designed with the help of protein database analysis software. The solid-phase peptide synthesis method is adopted to synthesize peptides separately and couple same to carrier proteins to complete the preparation of antigens. The animals are immunized, and the best antigenic epitopes are selected through experiments, such that a rabbit anti-Lactoferrin polyclonal flow cytometry antibody is successfully prepared with strong specificity, high sensitivity, and desirable stability. The present disclosure provides high-quality antibodies for scientific researchers, and opens up new ideas for flow cytometry antibody research and development.

The above are preferred examples of this application, but the protection scope of this application is not limited thereto. Therefore, all equivalent changes made in accordance with the structure, shape, and principle of this application shall fall within the protection scope of this application.

What is claimed is:

1. A Lactoferrin polypeptide fragment, wherein the Lactoferrin polypeptide fragment has an amino acid sequence shown in SEQ ID NO: 1.

2. A preparation method of the Lactoferrin polypeptide fragment according to claim 1, comprising the following steps:
S1, activation: obtaining a 9-fluorenylmethoxycarbonyl-protected amino acid (Fmoc-AA), and activating a carboxyl group of the Fmoc-AA to obtain a carboxyl-activated Fmoc-AA;
S2, ligation of a resin: subjecting the carboxyl-activated Fmoc-AA obtained in step S1 to a reaction with a P-hydroxymethylphenoxymethyl polyethylene resin (HMP resin) to obtain an Fmoc-AA-ligated resin;
S3, deprotection: removing an Fmoc protecting group of the Fmoc-AA-ligated resin prepared in step S2 through deprotection, such that an amino group of a remaining amino acid is activated to obtain an amino-activated resin;
S4, conducting coupling: repeating step S1 to prepare a new carboxyl-activated Fmoc-AA, and coupling the amino-activated resin prepared in step S3 with the new carboxyl-activated Fmoc-AA to obtain a new amino acid-coupled and Fmoc protecting group-containing resin;
S5, preparation of a polypeptide resin: conducting a process of repeating step S3 to remove the Fmoc protecting group from the new amino acid-coupled and Fmoc protecting group-containing resin prepared in step S4 and then repeating step S4 to couple a new amino acid to a resulting product, repeating S1 to S4, and repeating step S3 to remove the Fmoc protecting group until a target Lactoferrin polypeptide fragment-ligated resin is obtained; and
S6, conducting separation and purification: separating a resin from the target Lactoferrin polypeptide fragment-ligated resin prepared in step S5 to obtain a crude Lactoferrin polypeptide fragment, and purifying the crude Lactoferrin polypeptide fragment to obtain the Lactoferrin polypeptide fragment; wherein
the amino acids in steps S1 to S5 are added in an order of cysteine, alanine, leucine, cysteine, glutamate, threonine, asparagine, aspartate, asparagine, phenylalanine, leucine, leucine, asparagine, and lysine.

3. The preparation method of the Lactoferrin polypeptide fragment according to claim 2, wherein synthesis in steps S1 to S5 is completed using an automated polypeptide synthesizer.

4. The preparation method of the Lactoferrin polypeptide fragment according to claim 2, wherein the Fmoc-AA prepared by ligating an amino acid to an Fmoc protecting group through a protecting group reaction in step S1 has a structural formula as follows:

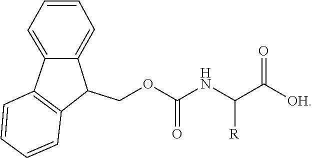

5. The preparation method of the Lactoferrin polypeptide fragment according to claim 2, wherein a process of activating the carboxyl group in step S1 comprises subjecting the Fmoc-AA to a reaction with N,N-dicyclohexylcarbodiimide (DCC) and 1-hydroxybenzotriazole (HOBT), and has a reaction formula as follows:

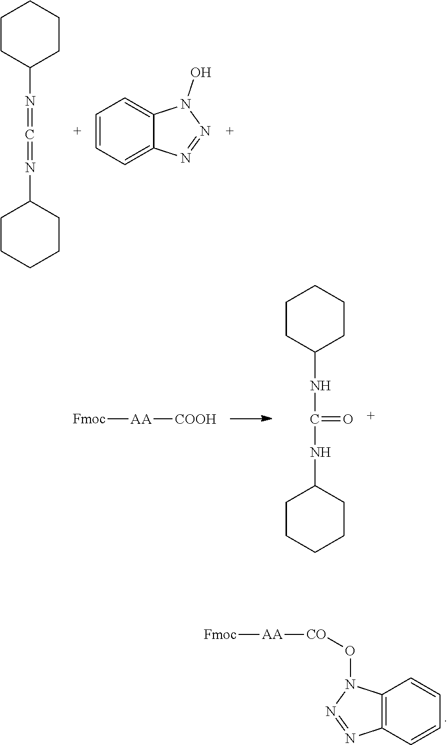

6. The preparation method of the Lactoferrin polypeptide fragment according to claim 2, wherein a process of subjecting the carboxyl-activated Fmoc-AA to the reaction with the HMP resin in step S2 is conducted in the presence of dimethylaminopyridine (DMAP), and has a reaction formula as follows:

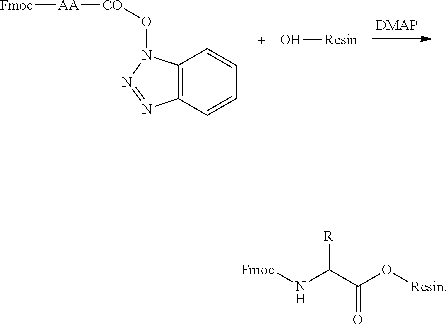

7. The preparation method of the Lactoferrin polypeptide fragment according to claim 2, wherein a process of removing the Fmoc protecting group in step S3 is conducted under the action of Piperidine, and has a reaction formula as follows:

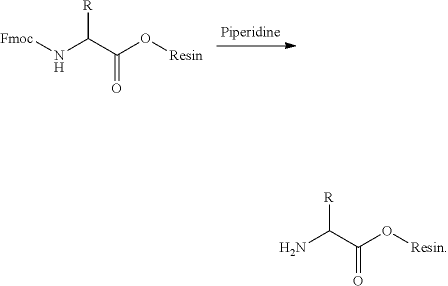

8. The preparation method of the Lactoferrin polypeptide fragment according to claim 2, wherein the coupling in step S4 refers to a coupling reaction with a reaction formula as follows:

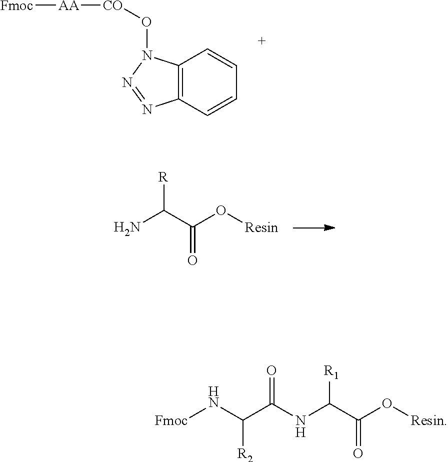

9. The preparation method of the Lactoferrin polypeptide fragment according to claim 2, wherein a process of separating the resin from the target Lactoferrin polypeptide fragment-ligated resin in step S6 comprises adding trifluoroacetic acid (TFA) combined with a scavenger mixed with 1,2-ethanedithiol (EDT), thioanisole, and water to allow a reaction with the target Lactoferrin polypeptide fragment-ligated resin to separate the target Lactoferrin polypeptide fragment from the resin.

10. The preparation method of the Lactoferrin polypeptide fragment according to claim 9, wherein after the target Lactoferrin polypeptide fragment and the resin are separated, the resin is removed by filtration while the scavenger is removed by vacuum distillation; and a resulting residue is subjected to water dissolution and extraction in sequence to obtain the crude Lactoferrin polypeptide fragment.

11. The preparation method of the Lactoferrin polypeptide fragment according to claim 10, wherein an extractant for the extraction is diethyl ether.

12. The preparation method of the Lactoferrin polypeptide fragment according to claim 2, wherein the crude Lactoferrin polypeptide fragment in step S6 is purified by high-performance liquid chromatography (HPLC).

13. The preparation method of the Lactoferrin polypeptide fragment according to claim 12, wherein the HPLC comprises:
chromatographic column: C₁₈ 25 mm×250 mm;
mobile phase A: 0.1% TFA aqueous solution; mobile phase B: 0.1% TFA dissolved in 60% acetonitrile;
detection wavelength: 214 nm;
flow rate: 10 mL/min; and
elution gradient: 20% to 60% of the mobile phase B for a total of 30 min.

14. An anti-Lactoferrin antibody prepared using the Lactoferrin polypeptide fragment according to claim 1, wherein the anti-Lactoferrin antibody is prepared by: coupling the Lactoferrin polypeptide fragment to a carrier protein, conducting rabbit immunization, collecting rabbit blood when a specific immunoglobulin (IgG) concentration in a rabbit serum reaches a peak, and then separating and purifying the rabbit serum for the anti-Lactoferrin antibody.

15. The anti-Lactoferrin antibody according to claim 14, wherein the carrier protein is selected from the group consisting of hemocyanin and keyhole limpet hemocyanin.

16. The anti-Lactoferrin antibody according to claim 14, wherein the rabbit immunization is conducted by basal immunization and/or multiple booster immunizations.

17. A method of use of the Lactoferrin polypeptide fragment according to claim 1 in preparation of an anti-Lactoferrin flow cytometric antibody and in preparation of a Lactoferrin detection product.
